(19) United States
(12) Patent Application Publication
(10) Pub. No.: US 2016/0051391 A1
(43) Pub. Date: Feb. 25, 2016

(54) CERVICAL SPLINTS AND METHODS FOR MAKING THE SAME

(71) Applicant: Bruce P. Abbott, Dartmouth, MA (US)

(72) Inventor: Bruce P. Abbott, Dartmouth, MA (US)

(21) Appl. No.: 14/829,862

(22) Filed: Aug. 19, 2015

Related U.S. Application Data
(60) Provisional application No. 62/038,916, filed on Aug. 19, 2014.

(51) Int. Cl. A61F 5/055; A61B 5/00

(52) U.S. Cl. CPC A61F 5/055; A61B 5/4824; A61F 2240/001

(57) ABSTRACT

A cervical splint assembly is provided for a person to alleviate headaches or cervical conditions. The cervical splint includes a splint assembly having a first end, a second end, and a body extending between the first end and the second end. The body has a front and a back and is formed of compressible material and sized to fit between a chin and a top of a chest of the person. A strap has a first end and a second end selectively and adjustably connected together. The strap wraps around the front of the body such that when the splint assembly is secured to a neck of the person using the cervical splint, the splint assembly provides support for a chin of the person, restricts flexion of a neck of the person, and the first and second ends are aligned approximately below earlobes of the person.

### CROSS-REFERENCE TO RELATED APPLICATION

This application claims priority to U.S. Provisional Patent Application No. 62/038,916, filed Aug. 19, 2014, which is incorporated herein by reference.

### BACKGROUND

1. Technical Field

The present disclosure relates generally medical devices and more particularly to cervical splints to aid in the treatment of headaches and cervical conditions (such as spondylosis, radiculopathy, disk disease, myelopathy, and neck sprain) where undesired neck position may cause, trigger or exacerbate symptoms.

2. Background of the Related Art

Headache disease management is medically relevant because: (1) headache is the number one complaint for which patients seek medical advice; (2) as of 2002, there were 46 million chronic headache suffers in the US and over 700 million worldwide as reported by Neurology, March 2002, vo, 58, no. 6; many more exist today; (3) lost days to headache account for billions of dollars yearly; (4) no other neck splint device exists to treat these conditions; and (5) primary care doctors and mid-level providers who treat 94% of chronic headache suffers have on average 7 minutes per visit to assess and manage the over 30 frequent causes of chronic headache. Results are often unsatisfactory.

The two most common types of headache worldwide are tension type headache and migraine. 88% of females will experience tension type headache and 25% of females have migraine. 69% of males will experience tension type headache and 7.5% of males have migraine.

There are a variety of headache types, each having different symptoms and treatments. One particular type of headache is a positional headache, with is primarily caused by the patient's sleeping position but may also be caused by workspace ergonomics and poor posture. For instance, some office jobs may have the patient chin down, placing strain at the back of the neck.

Many attempts have been made to address providing neck support with cervical collars. For example, see U.S. Pat. No. 6,458,090, U.S. Pat. No. 7,090,652, U.S. Pat. No. 3,921,626. Additionally, improper positioning of the head can exacerbate sleep apnea and snoring. Further neck supports have been developed to particularly address snoring and sleep apnea. For example, see U.S. PGPUB No. 2012/234330 and U.S. Pat. No. 4,366,815.

### SUMMARY

In view of the above, there is a need for effective treatment of position-induced headaches, snoring, and/or sleep apnea and the like. Preferably, the cervical splint is cost effective. Further, an efficient method to produce cervical splint is also desired. Further, it is desirable to have a cervical splint that can be easily applied, adjusted and customized by the user. The subject technology for cervical splints can also be used in conjunction with headache diagnosis and analysis tools as disclosed in U.S. PGPUB No. 2015/0220694 published on Aug. 6, 2015.

In one embodiment, the subject technology is directed to a cervical splint that provides thin support thereby relieving stress on the back of the neck and shoulders to the patient. As a result, the patient's headache symptoms are relieved. Similarly, other cervical conditions (such as spondylosis, radiculopathy, disk disease, myelopathy, neck sprain, sleep apnea, snoring and the like) are also alleviated. The cervical splint includes an arcuate molded piece of foam that supports the users chin. The foam splint is held in place by a strap with fasteners, such as hook and loop fasteners.

Another embodiment of the subject technology is directed to a cervical splint assembly for a person to alleviate headaches or cervical conditions. The cervical splint includes a splint assembly having a first end, a second end, and a body extending between the first end and the second end. The body has a front and a back and is formed of compressible material and sized to fit between a chin and a top of a chest of the person. A strap has a first end and a second end selectively and adjustably connected together. The strap wraps around the front of the body such that when the splint assembly is secured to a neck of the person using the cervical splint, the splint assembly provides support for a chin of the person, restricts flexion of a neck of the person, and the first and second ends are aligned approximately below earlobes of the person.

The body may have a top and a bottom between the front and the back, the top being relatively bulbous with respect to the bottom so that the back of the body approximates a profile of a person's neck and chin to fit snugly there against. The cervical splint assembly may also have a spacer selectively attached to the bottom to adjust fit for various neck lengths. The strap can be formed of an elastic material, initially at least 46 cm in length, and fabricated from a material that can be easily cut to remove excess. The cervical splint assembly may also include a removable pouch covering the body. In one embodiment, the body is approximately 18 cm long, 2 cm thick, and 6 cm tall.

The cervical splint may also be fabricated by following the steps of: providing an elongated solid cylinder having a length along an axis, wherein the length is approximately half of a circumference of the patient's neck; cutting the elongated solid cylinder lengthwise to form a first portion and a second portion, wherein the first portion has an elongated flat side opposing a curved side with both sides extending between a first end and a second end; cutting the first and second ends at an angle with respect to the flat side without substantially reducing the length of the curved side to form angled ends; and providing a strap with the first portion for selectively securing the first portion around a neck of the patient with the curved side adjacent the neck and under a chin of the patient so that the angled ends allow the strap to transition from the flat side to the neck without a significant gap.

The method may further include the steps of: cutting the first portion lengthwise to form a first shim portion; providing the first shim portion with the first portion so that the patient may selectively use the first shim portion to vary a thickness of the cervical splint; cutting the first portion lengthwise a second time to form a second shim portion; providing the second shim with the first portion and the first shim portion so that the patient may selectively use the first shim portion, the second shim portion or both of the first and second shim portions to vary a thickness of the cervical splint; enclosing at least one of the first portion, the first shim and the second shim in a fabric pouch; and attaching the strap to the first portion.

The method of fabrication may further include the steps of cutting the first and second ends of the second portion at an angle with respect to the flat side of the second portion without substantially reducing the length of the curved side of the second portion to form angled ends; providing a strap with the second portion for selectively securing the second portion around a neck of a patient with the curved side adjacent the neck and under a chin of the patient so that the angled ends allow the strap to transition from the flat side to the neck without a significant gap; cutting the second portion lengthwise to form a first shim portion; and providing the first shim portion of the second portion with the second portion so that the patient may selectively use the first shim portion of the second portion to vary a thickness of the second portion, wherein the second portion has an elongated flat side opposing a curved side with both sides of the second portion extending between a first end and a second end. The method may further include the step of cutting the strap to remove excess.

Another embodiment of the subject technology is a cervical splint including a strap for securing the cervical splint to a neck of a patient and an elongated body. The body has a first end opposing a second end, each end being angled so that when the strap secures the elongated body to the neck, the strap smoothly transitions from the angled ends to directly contact the back of the neck while the elongated body supports a chin of the patient. The elongated body may have one or more flat sides. An elongated band can couple to the elongated body for retaining the elongated body in a profile similar to that of the patient's neck, wherein the elongated band is flexible to allow for adjustment and contouring.

Preferably, diagnosis of the patient results in use of the subject technology. The diagnosis may be in accordance with the teachings of U.S. PGPUB No. 2015/0220694. For example, use of the subject technology could include the steps of diagnosing and treating headaches using a computer server having diagnostic software tool operative to: collect detailed medical symptoms information from a patient of the patient's headaches; analyze the answers from the patient; create a report of headache conditions and comorbidities based on the detailed medical symptoms information collected from the patient via; transmit the report to a health care provider; review the report to determine an optimal treatment plan for the patient; and creating a treatment plan for the patient based on the report. The treatment plan preferably includes use of a cervical splint as taught herein.

It should be appreciated that the subject technology can be implemented and utilized in numerous ways, including without limitation as a process, an apparatus, a system, a device, a method for applications now known and later developed. These and other unique features of the system disclosed herein will become more readily apparent from the following description and the accompanying drawings.

### BRIEF DESCRIPTION OF THE DRAWINGS

These and other features, aspects, and advantages of the present invention will become better understood with reference to the following description, appended claims, and accompanying drawings.

FIG. 1 is a perspective view of a cervical splint deployed on a person in accordance with the subject technology.

FIG. 2 is an exploded view of a cervical splint in accordance with the subject technology.

FIG. 3 is a cross-sectional top view of a cervical splint in accordance with the subject technology.

FIG. 4 is a perspective view of another cervical splint body having optional portions for adjusting a size of the cervical splint in accordance with the subject technology.

FIG. 5 is a perspective view of still another cervical splint body having slots for improved fit in accordance with the subject technology.

FIG. 6 is a perspective view of another contoured cervical splint body in accordance with the subject technology.

FIG. 7 is a cross-sectional view of the cervical splint body of FIG. 6.

FIGS. 8-12 are a cervical splint body in perspective, front, back, bottom/top, and side view, respectively.

### DESCRIPTION OF THE PREFERRED EMBODIMENT

The subject technology overcomes many of the prior art problems associated with prevention and treatment of headaches by using neck supports. The subject technology is also particularly useful for treating headaches diagnosed in accordance with the teaching of U.S. PGPUB No. 2015/0220694 published on Aug. 6, 2015. The advantages, and other features of the system disclosed herein, will become more readily apparent to those having ordinary skill in the art from the following detailed description of certain preferred embodiments taken in conjunction with the drawings which set forth representative embodiments of the present disclosure and wherein like reference numerals identify similar structural elements.

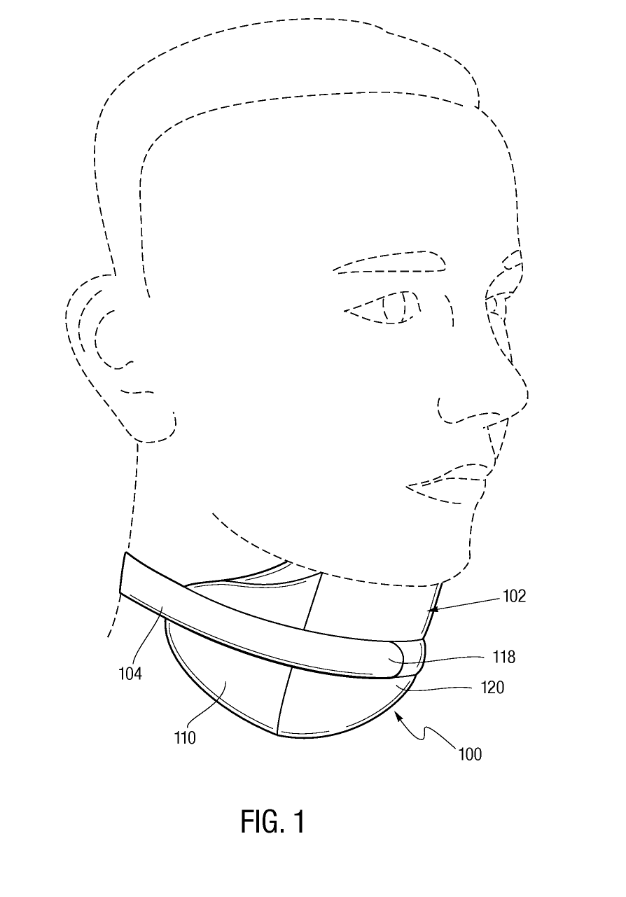

Referring now to the FIG. 1, there is shown a perspective view of a cervical splint 100 deployed on a patient in accordance with the subject technology. The cervical splint 100 includes a support assembly 102 coupled under the patient's chin and against the patient's neck to properly position and support the head of the patient. In a preferred embodiment, the support assembly 102 does not encircle the patient's neck. However, the support assembly 102 can full encircle the patient's neck, cover at least half or three quarters of the patient's neck, or simply cover a small area under the patient's chin. In one embodiment, the support assembly 102 is U-shaped and sized to wrap from below one earlobe to the other earlobe. The cervical splint 100 is desirably comfortable to wear whether use occurs during daily activity or during rest. The support assembly 102 is retained in place by a strap 104. Preferably, the strap 104 is elastic to provide some compression and a snug fit of the support assembly 102. The strap 104 is also long enough so that the ends 116, 118 can be secured together by the patient in front and wrap around the support assembly twice.

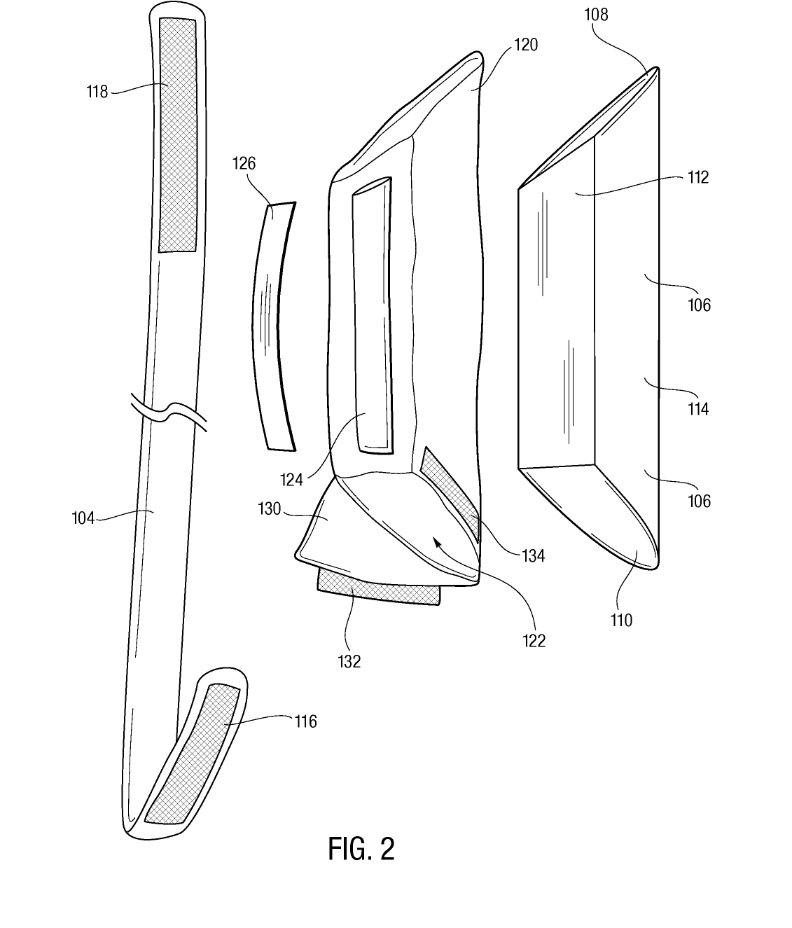

Referring now to FIG. 2, an exploded view of the cervical splint 100 is shown. The support assembly 102 includes a body 106 having a first end 108 and a second end 110. The body 106 has a flat front side 112 opposing a curved back side 114. The body 106 is formed of compressible material and sized to fit between a chin and a top of a chest of the person. The support assembly 102 provides support for a chin of the person, restricts flexion of the person's neck. Preferably, the body 106 is soft foam material. As shown, the body 106 is relatively straight. In an alternative embodiment, the body 106 is molded to be U-shaped. A length of the support assembly 102 is sized so that a portion of the person's neck is encircled by the support assembly 102. Preferably, the first and second ends 108, 110 are aligned approximately below earlobes of the person, respectively. The ends 108, 110 are also angled so that the transition of the strap 104 from the support assembly 102 to the neck is fully supported without significant gapping. In one embodiment, the body is approximately 18 cm long, 2 cm thick, and 6 cm tall.

The strap 104 has the first end 116 and the second end 118. The ends 116, 118 may be selectively secured together to wrap around the support assembly 102 to secure the support assembly 102 to the person's neck. In one embodiment, the strap 104 is formed at least 46 cm in length and fabricated from a material that can be easily cut by the patient to customize the length and remove excess.

Still referring to FIG. 2, the ends 116, 118 include hook and loop fabric so that not only are the ends 116, 118 attachable but the tightness of the strap 104 may be adjusted to enhance fit of the support assembly 102 to the patient's neck. Alternatively, the strap 104 may have a buttons and buttonholes to couple the end 116, 118, a buckle, clips, snaps or like arrangements instead of hook and loop fabric.

For protection and added softness, the body 106 is enclosed in a cloth sleeve or pouch 120. The pouch 120 has an opening 122 so that the body 106 can be removed for cleaning of the body 106 and pouch 120 as desired. The opening 122 is closed by a flap 130 having a hook and loop closure portion 132 that matches with a corresponding closure portion 134. The pouch 120 may also have hook and loop fabric that contacts corresponding hook and look fabric on the strap 104 to selectively secure the pouch 120 and strap 104 together. A belt loop arrangement on the pouch 120 may alternatively couple the pouch 120 and strap 104 together.

The pouch 120 also includes an elongated pocket 124 for receiving a shape band 126. The body 106 is normally straight. By coupling the shape band 126 to the body 106, the body 106 can be formed into U-shape. The shape band 126 can be a pliable material that can be bent into a desired shape for a custom fit. For example, the shape band 126 can be soft metal. The shape band 126 could also be plastic and preshaped in a U-shape. The pouch 120 may also include a decorative element such as initial, an embroidery pattern and the like.

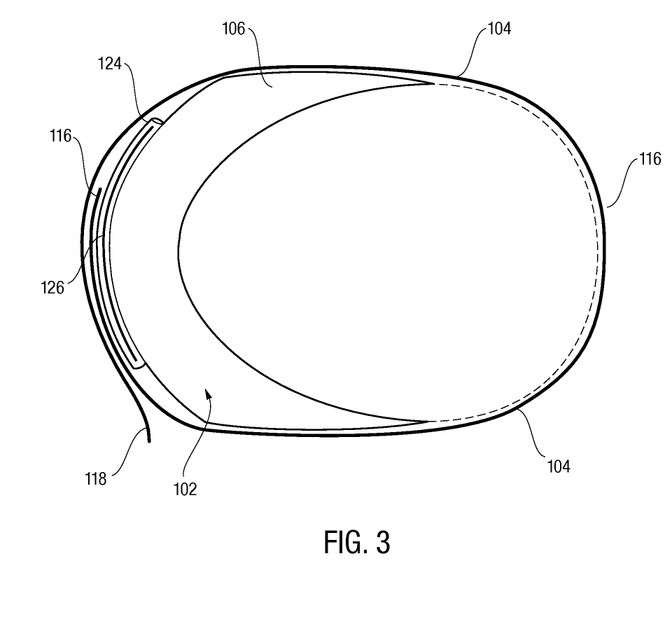

Referring now to FIG. 3, a cross-sectional view of the cervical splint 100 when assembled is shown. As can be seen, the shape band 126 helps to conform the body 106 into a U-shape. The strap 104 further wraps around the body 106 and band 126 to further help the support assembly 102 snugly fit upon the patient and, thereby, comfortably support the patient's head in a proper position and provide effective treatment. Although excessively long for most users, the strap 104 is easily customizable while wearing by securing the end 116, 118 in front of the patient and cutting the excess strap. in an alternative embodiment, the cervical splint 100 consists only of the body 106 and the strap 104. Typically, a person's neck varies up to 24 inches in circumference. Thus, the strap 104 may be more than 24 inches long for double wrapping or to provide ample overlap.

In order to fabricate the body 106, it is advantageous to start with an elongated solid cylinder having a length along an axis, wherein the length is approximately half of a circumference of the patient's neck. The elongated solid cylinder can be centrally cut lengthwise to form a first portion and a second portion. Each portion can be used to make a separate body 106. At this point in the manufacture, each portion has an elongated flat side opposing a curved side with flat end surfaces that are substantially perpendicular to the flat side.

In order to form the angled ends 108, 110, the flat end surfaces are cut at an angle with respect to the flat side. In order to use the material efficiently, the angled cuts do not substantially reduce the length of the curved side. As a result of the angled ends, when the strap 104 secures the body 106 around a patient's neck, the strap 104 transitions from the flat front 112 to the neck without a significant gap.

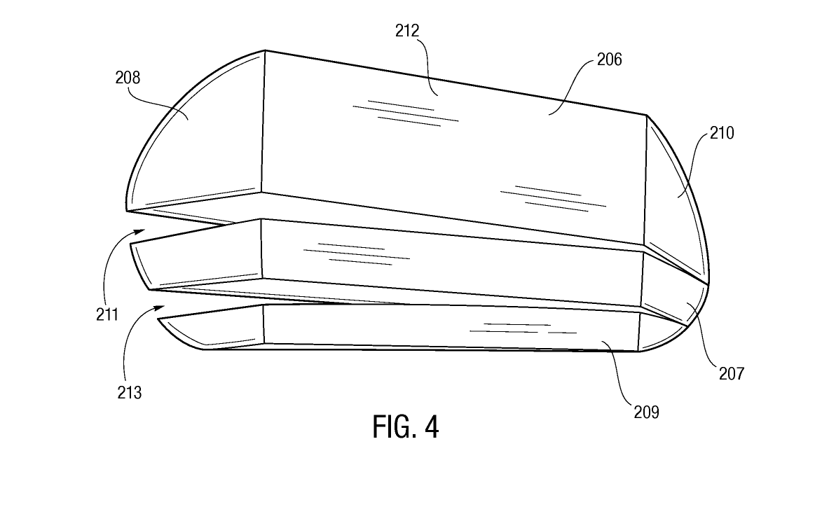

Referring now to FIG. 4, a perspective view of another body 206 for a cervical splint is shown. Similar elements to those described in connection with above-described embodiments are indicated with the like reference numbers. Many structural elements are very similar to those of the foregoing embodiments and, thus, are not further described herein. The primary differences between the cervical splint body 206 and the cervical splint body 106 are the first and second optional body portions 207, 209 for adjusting a size of the body 206 to accomplish an improved fit upon the patient.

The optional body portions 207, 209 are formed by cutting partial lengthwise slits 211, 213, respectively, in the body 206. If the spacing of the slits 211, 213 is such that optional body portion 207 is thicker than optional body portion 209. The patient may utilize the body 206 with both optional body portions 207, 209. The patient may also remove one or both optional body portions 207, 209 to reduce the size of the body 206. Once removed, optional body portion 209 can simply be set against body 206 inside the pouch (not shown) to create an additional size.

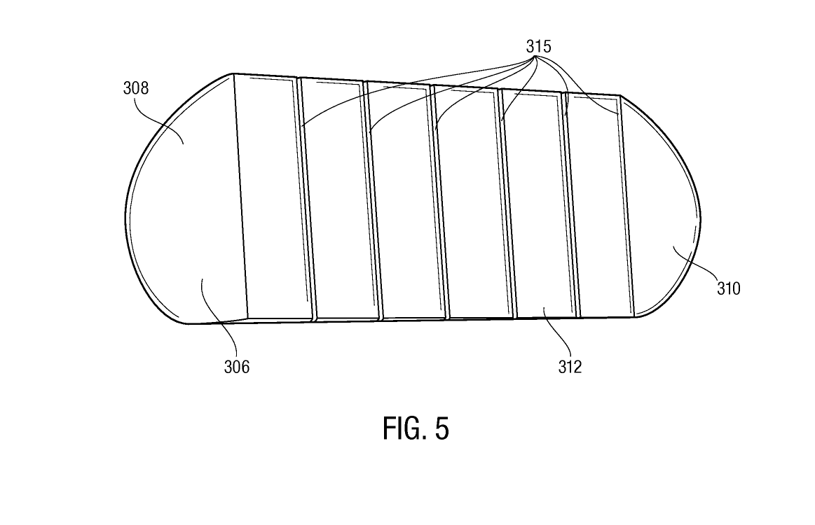

Referring now to FIG. 5, is an exploded view of still another cervical splint body 306 is shown. Similar elements to those described in connection with above-described embodiments are indicated with the like reference numbers. The primary differences of the cervical splint body 306 are the slots 315 for improved flexibility and fit. The slots 315 are cut substantially perpendicular to the length of the body 306 all around the body 306 but not completely through the body 306. Alternatively, these slots may be only on one side or on multiple sides of the body 306. There may also be a plurality of closely spaced slots on the ends 308, 310 so that various portions can be easily removed to adjust a length of the body 306.

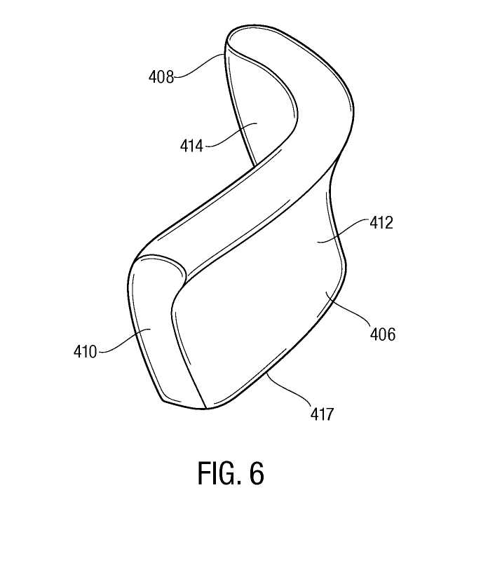

Referring now to FIGS. 6 and 7, still another cervical splint body 406 is shown in perspective and cross-sectional views. The cervical splint body 406 may come in different sizes and/or have attachable spacers so that the size can be adjusted. It is envisioned that spacers can be coupled to a bottom 417 of the cervical splint body 406 to fit various neck lengths. In one embodiment, the cervical splint body 406 is approximately eighteen centimeters long as measured on the front, arcuate surface, two centimeters thick, or deep, and six centimeters high. By providing the cervical splint body 406 with a profile that more closely matches the patient's neck area, an improved fit is accomplished. It is envisioned that the cervical splint body 406 can be fabricated by 3D printing. The ultimate pattern for 3D printing can be complimentary to a 3D image of the patient's neck area.

Generally, the patient wears the cervical splint by positioning the body against their neck, between the chin and top of the chest. The strap adheres to the front of the splint. The splint is secured in place by wrapping the strap about the neck and fastening the ends of the strap together to a desired tightness. When adjusted properly on the neck, the cervical splint restricts flexion of the neck by limiting movement of the chin onto the chest. As a result, cervical para spinal muscles are rested and tension transmitted to the scalp and muscles of mastication is reduced. Headaches related to neck muscle tension, or triggered by neck muscle tension—cervicogenic headache, migraine headache and tension headache—are relieved by this immobilization and rest.

Another application of the subject technology is to treat symptoms of cervical spondylosis (e.g., wear and tear), cervical myelopathy (e.g., spinal cord kinking), and cervical radiculopathy (e.g., nerve root pinching) by splinting the neck to prevent excessive flexion which aggravates wear on bony structures and disks and muscles and ligaments. Further, splinting a patient's neck in accordance with the subject technology can prevent ischemic insult (e.g., lack of blood flow) to the spinal cord and nerve roots which are pulled up against bony structures to cause kinking.

Preferably, the cervical splint is worn when sleeping, which is the most common time the neck flexes. Some environments may cause more irritation. For instance, like falling asleep in an airplane seat and the like, where proper head support is not available. The cervical splint can be worn when awake when the patient is doing chin down work like typing, to provide additional support. As can be seen, the cervical splint treats the inciting injury, creates an environment for healing, and prevents re-injury once a person has improved.

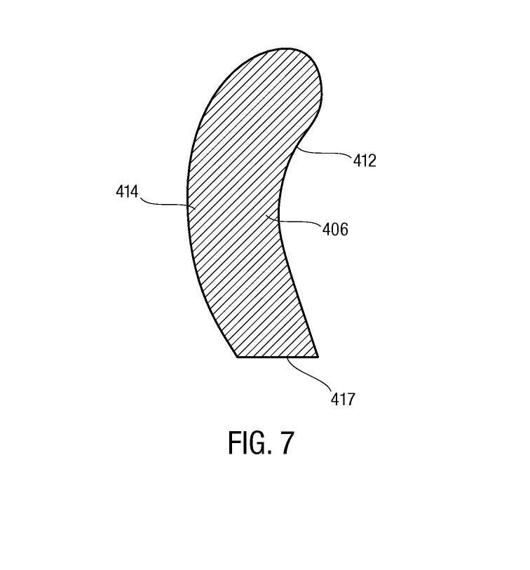

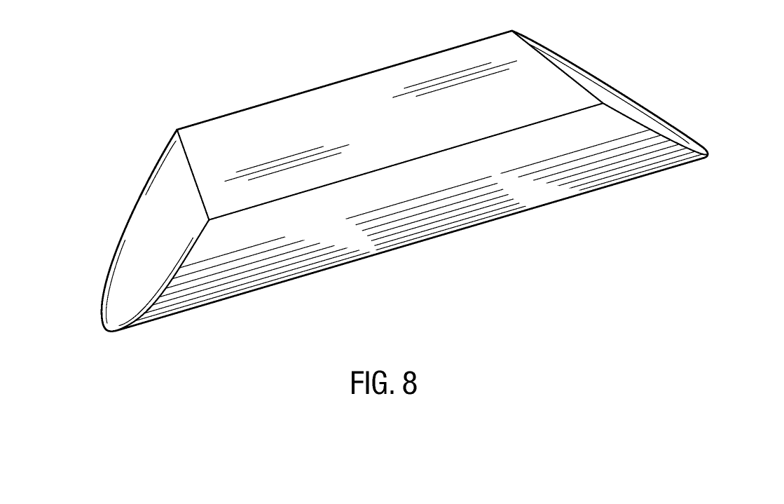

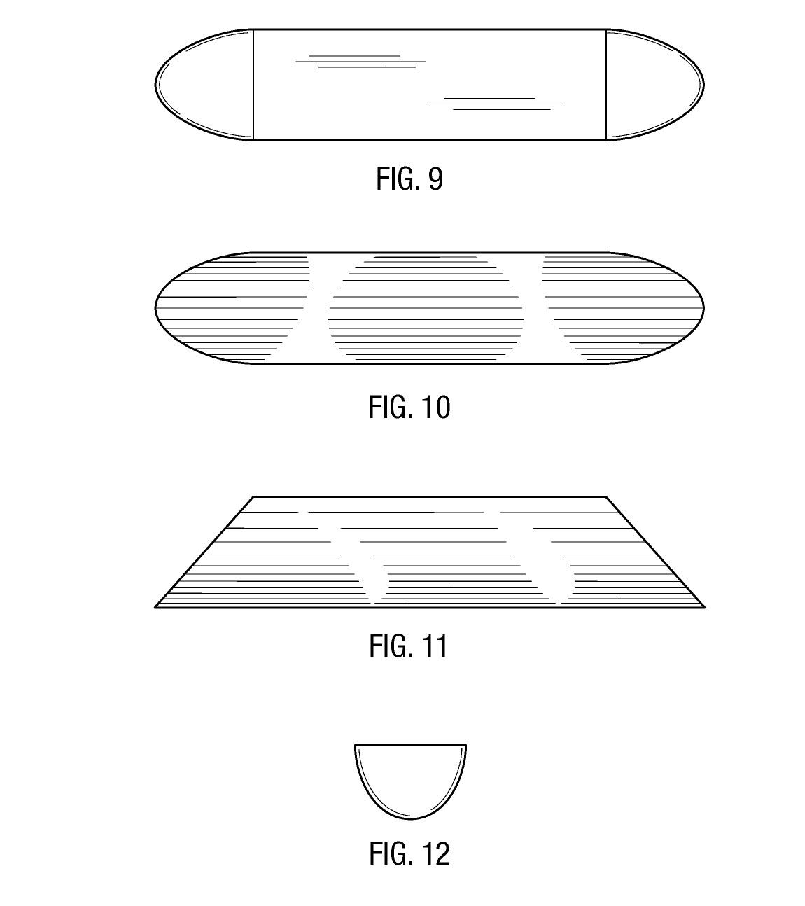

Referring now to FIGS. 8-12, a cervical splint body 106 is shown in perspective, front, back, bottom/top, and side view, respectively. As would be appreciated, the bottom and top views as well as each side view would be similar so that separate figures for each is unnecessary. The ends 108, 110 may be cut an any angle and, thus, are shown to highlight details for the purpose of illustrating portions of the cervical splint body and form an optional part of the design. Similarly, in FIG. 12, the design is shown as a semi-circle but other shapes would be possible including square (e.g., by cutting a rectangle in half), rectangular (e.g., by cutting a square in half), triangular , by cutting a square in half rectangularly) and the like.

It will be appreciated by those of ordinary skill in the pertinent art that the functions of several elements may, in alternative embodiments, be carried out by fewer elements, or a single element. Similarly, in some embodiments, any functional element may perform fewer, or different, operations than those described with respect to the illustrated embodiment. Also, functional elements (e.g., straps, pouches, bands and the like) shown as distinct for purposes of illustration may be incorporated within other functional elements in a particular implementation. All such modifications and changes are intended to be within the scope of the present invention.

All patents, patent applications and other references disclosed herein are hereby expressly incorporated in their entireties by reference. While the subject technology has been described with respect to preferred embodiments, those skilled in the art will readily appreciate that various changes and/or modifications can be made to the subject technology without departing from the spirit or scope of the invention as defined by the appended claims.

What is claimed is:

1. A cervical splint assembly for a person to alleviate headaches or cervical conditions comprising:
a splint assembly having a first end, a second end, and a body extending between the first end and the second end, wherein the body has a front and a back, the body is formed of compressible material and sized to fit between a chin and a top of a chest of the person;
a strap has a first end and a second end selectively and adjustably connected together, the strap wraps around the body such that when the splint assembly is secured to a neck of the person using the cervical splint, the splint assembly provides support for a chin of the person, restricts flexion of a neck of the person, and the first and second ends are aligned approximately below earlobes of the person.

2. A cervical splint assembly as recited in claim 1, wherein the body has a top and a bottom between the front and the back, the top being relatively bulbous with respect to the bottom so that the back of the body approximates a profile of a person's neck and chin to fit snugly there against.

3. A cervical splint assembly as recited in claim 2, further comprising a spacer selectively attached to the bottom to adjust fit for various neck lengths.

4. A cervical splint assembly as recited in claim 1, wherein the strap is formed of an elastic material, initially at least 24 inches in length, and fabricated from a material that can be easily cut to remove excess.

5. A cervical splint assembly as recited in claim 1, further comprising: a removable pouch covering the body; and a decorative element on the pouch.

6. A cervical splint assembly as recited in claim 5, wherein a center of the strap attached to the front of the body and ends of the strap are secured together at the front of the body.

7. A cervical splint assembly as recited in claim 1, wherein the body is approximately 18 cm long, 2 cm thick, and 6 cm tall.

8. A method of manufacturing a cervical splint sized to a neck of a patient comprising the steps of:
providing an elongated solid cylinder having a length along an axis, wherein the length is approximately half of a circumference of the patient's neck;
cutting the elongated solid cylinder lengthwise to form a first portion and a second portions wherein the first portion has an elongated flat side opposing a curved side with both sides extending between a first end and a second end;
cutting the first and second ends at an angle with respect to the flat side without substantially reducing the length of the curved side to form angled ends; and
providing a strap with the first portion for selectively securing the first portion around a neck of the patient with the curved side adjacent the neck and under a thin of the patient so that the angled ends allow the strap to transition from the flat side to the neck without a significant gap.

8. A method as recited in claim 8, further comprising the steps of:
cutting the first portion lengthwise to form a first shim portion; and
providing the first shim portion with the first portion so that the patient may selectively use the first shim portion to vary a thickness of the cervical splint.

10. A method as recited in claim 9, further comprising the steps of:
cutting the first portion lengthwise a second time to form a second shim portion; and
providing the second shim with the first portion and the first shim portion so that the patient may selectively use the first shim portion, the second shim portion or both of the first and second shim portions to vary a thickness of the cervical splint.

11. A method as recited in claim 10, further comprising the step of enclosing at least one of the first portion, the first shim and the second shim in a fabric pouch.

12. A method as recited in claim 8, further comprising the step of cutting at least one slot in the first portion perpendicular to the axis.

13. A method as recited in claim 12, wherein the at least one slot is a first plurality of parallel slots and a second plurality of parallel slots in an opposing location with respect to the first plurality of parallel slots.

14. A method as recited in claim 8, further comprising the steps of attaching the strap to the first portion; and cutting the strap to remove excess once the cervical splint is on the neck.

15. A method as recited in claim 8, wherein the second portion has an elongated flat side opposing a curved side with both sides of the second portion extending between a first end and a second end, and
further comprising the steps of:
cutting the first and second ends of the second portion at an angle with respect to the flat side of the second portion without substantially reducing the length of the curved side of the second portion to form angled ends;
providing a strap with the second portion for selectively securing the second portion around a neck of a patient with the curved side adjacent the neck and under a chin of the patient so that the angled ends allow the strap to transition from the flat side to the neck without a significant gap;
cutting the second portion lengthwise to form a first shim portion; and
providing the first shim portion of the second portion with the second portion so that the patient may selectively use the first shim portion of the second portion to vary a thickness of the second portion.

16. A method as recited in claim 8, further comprising the steps of diagnosing and treating headaches using a computer server having diagnostic software tool operative to: collect detailed medical symptoms information from a patient of the patient's headaches; analyze the answers from the patient; create a report of headache conditions and comorbidities based on the detailed medical symptoms information collected from the patient via; transmit the report to a health care provider; review the report to determine an optimal treatment plan for the patient; and creating a treatment plan for the patient based on the report.

17. A cervical splint comprising:
a strap for securing the cervical splint to a neck of a patient; and
an elongated body having a first end opposing a second end, each end being angled so that when the strap secures the elongated body to the neck, the strap smoothly transitions from the angled ends to directly contact the back of the neck while the elongated body supports a chin of the patient.

18. A cervical splint as recited in claim 17, wherein the elongated body has a flat side and is removably enclosed in fabric.

19. A cervical splint as recited in claim 17, wherein the elongated body is fabricated by 3D printing based upon a 3D pattern based upon an area of the neck of the patient.

20. A cervical splint as recited in claim 17, further comprising an elongated band coupled to the elongated body for retaining the elongated body in a profile similar to that of the patient's neck, wherein the elongated band is flexible to allow for adjustment.
